(19) United States
(12) Patent Application Publication
(10) Pub. No.: US 2026/0104412 A1
(43) Pub. Date: Apr. 16, 2026

(54) COVALENT TETHERING OF PORTAL PROTEIN INTO SOLID-STATE NANOPORES

(71) Applicant: Northeastern University, Boston, MA (US)

(72) Inventors: Meni Wanunu, Sharon, MA (US); Mehrnaz Mojtabavi, San Diego, CA (US)

(21) Appl. No.: 18/863,462

(22) PCT Filed: May 24, 2023
(86) PCT No.: PCT/US2023/067412

§ 371 Date: Nov. 6, 2024

Related U.S. Application Data
(60) Provisional application No. 63/365,259, filed on May 24, 2022.

(51) Int. Cl. G01N 33/543; G01N 33/487; G01N 33/68

(52) U.S. Cl. CPC G01N 33/54366; G01N 33/48721; G01N 33/6872

(57) ABSTRACT

Sensors and related methods of making and using sensors are disclosed. The sensor include a portal protein, such as G20c derived from the bacteriophage Thermits thermophilus, covalently bound to a thiol-silane surface of a solid-state membrane having one or more nanopores. The sensors can be used for protein and/or nucleotide sequencing and for small-molecule detection.

### RELATED APPLICATION

This application claims the benefit of U.S. Provisional Application No. 63/365,259, filed on May 24, 2022. The entire teachings of the above application is incorporated herein by reference.

### INCORPORATION BY REFERENCE OF MATERIAL IN XML

This application incorporates by reference the Sequence Listing contained in the following eXtensible Markup Language (XML) file being submitted concurrently herewith:
a) File name: 52002337001_Sequence_Listing.xml; created May 24, 2023, 8,790 Bytes in size.

### BACKGROUND

Studying biomolecular systems at the single-molecule level has unleashed a new era in probing the structure and dynamics of biomolecules with unprecedented detail. Nanopore sensors have shown unprecedented performance in recognition and quantification of various analytes and sequencing of individual molecules. Progress in nanopore technology in the last decade has resulted in the development of two platforms: solid-state nanopores and protein nanopores, with each platform having specific advantages in terms of performance/resolution, signal reproducibility, pore size/chemistry tunability, and chemical/physical robustness.

Protein nanopores typically require an organic support membrane (e.g., lipid bilayers or block-copolymers) that is significantly less robust than ceramic-based solid-state membranes (e.g., silicon nitride). On the other hand, no methods exist for reproducing solid-state nanopores in atomic detail, preventing progress in solid-state nanopore technology. Various reports have miniaturized the organic membrane areas in order to stabilize protein pores to achieve a more robust platform in which higher voltages can be applied, although ultimately the organic membrane stability determines the device's operating voltage range and duration. Combining the two types of pore systems to form hybrid pores has been achieved, although a shared limitation of both approaches is that the protein pore was not chemically linked to the solid-state pore, which results in a transient hybrid pore to which a limited voltage range can be applied.

Site-specific, reproducible, and uniformly oriented protein immobilization within a synthetic membrane in a manner that preserves the protein conformation and functionality requires an understanding of the membrane's surface chemistry, protein's properties, and the nature of their interaction to eliminate non-specific binding.

### SUMMARY

The G20c portal protein (SEQ ID NO: 1) was previously inserted into a lipid bilayer environment (unnatural for the portal protein) and later demonstrated voltage-induced “corking” of the portal into a solid-state nanopore. This protein has shown high stability and tunability. Disclosed herein is a novel type of hybrid nanopore system that covalently immobilizes the G20c portal protein within a chemically modified SiNₓ nanopore (e.g., a Si₃N₄ nanopore) to fabricate an ultra-stable hybrid nanopore that is functional at high voltages and confined in three dimensions. Application of this hybrid nanopore in sensing DNA molecules through free translocation and motor protein-mediated ratcheting at high voltages is described herein. The ability to apply high voltage in a protein-based nanopore platform can allow control over enzyme-based DNA movement, long-range DNA scanning, and further can be used for measurements of the pore resolution at voltages that are beyond the capabilities of traditional membrane-based pores. As shown herein, the present approach to producing hybrid nanopores by chemically linking protein to the solid-state pore constitutes the first step towards high-performance, high throughput array-based pore devices.

Described herein is a method of making a sensor. The method involves: a) reacting a membrane, which has a nanopore and a silicon nitride surface, with 2,2-dimethoxyl-1-thia-2-silacyclopentane, thereby forming a thiol-silane surface on the membrane comprising the nanopore; and b) contacting the membrane comprising the nanopore and the thiol-silane surface with a portal protein in the presence of Cu(phenanthroline)₂, thereby forming a disulfide bond between a cysteine residue of the portal protein and the thiol-silane surface of the membrane comprising the nanopore.

The disulfide bond can be between the cysteine residue of the portal protein and the thiol-silane surface of the nanopore portion of the membrane. Reacting the membrane having the nanopore and the silicon nitride surface with 2,2-dimethoxyl-1-thia-2-silacyclopentane can occur in the presence of a polar aprotic solvent, such as dicholoromethane.

The portal protein can be hydrophilic. The portal protein can be a G20c mutant having at least one amino acid substitution of an externally facing leucine to a cysteine. The portal protein can be a G20c mutant having at least one amino acid substitution from a negatively charged amino acid to a polar amino acid, such as an amino acid substitution from aspartic acid to asparagine. The portal protein can be a G20c mutant in which four aspartic acid residues are substituted with asparagine. The portal protein can include SEQ ID NO: 3.

The method can further include rinsing the membrane with solvent prior to step b).

The silicon nitride surface of the membrane can be on silicon dioxide.

The method can further include making a nanopore in the membrane by transmission electron microscopy. The nanopore diameter can be from 7 nm to 8 nm. The method include immersing the membrane in a mixture of sulfuric acid and hydrogen peroxide after making the nanopore in the membrane. Immersing the membrane can occur for 20 minutes to 30 minutes.

The method can further include washing the membrane with hot water after immersion in the mixture of sulfuric acid and hydrogen peroxide. Washing the membrane can occur for 20 minutes to 30 minutes.

The method can further include drying the membrane, such as drying the membrane by using N₂ and/or by baking the membrane at a temperature from 130° C. to 170° C. for duration from 3 minutes to 5 minutes.

Described herein is a sensor. The sensor can include a membrane having a nanopore with a portal protein through the nanopore. The membrane can be covalently bonded to the portal protein by a disulfide bond between a cysteine residue of the portal protein and a thiol-silane surface of the membrane. The thiol-silane surface of the membrane can be formed on a silicon nitride surface.

The portal protein can be hydrophilic. The portal protein can include a G20c mutant having at least one amino acid substitution of an externally facing leucine to a cysteine. The portal protein can include a G20c mutant having at least one amino acid substitution from a negatively charged amino acid to a polar amino acid, such as an amino acid substitution is from aspartic acid to asparagine. The portal protein can be a G20c mutant in which four aspartic acid residues are substituted with asparagine. The portal protein can include SEQ ID NO: 3.

The sensor can separate a first conductive liquid from a second conductive liquid. The sensor can include a tunnel that provides liquid communication between the first conductive liquid and second conductive liquid. The first conductive liquid medium can include at least one analyte, such as a polynucleotide, a polypeptide, or a combination thereof.

The portal protein can be stably bound to the membrane at applied voltages of at least 200 mV, at least about 300 mV, at least about 400 mV, at least about 500 mV, at least about 600 mV, or at least about 750 mV.

Described herein is a method for sensing an analyte. The method can include: a) providing a sensor that has a membrane, which has a thiol-silane surface and a nanopore, with a portal protein through the nanopore, wherein the membrane is covalently bonded to the portal protein by a disulfide bond between a cysteine residue of the portal protein and the thiol-silane surface of the membrane; b) providing a first conductive liquid and a second conductive liquid, wherein the portal protein comprises a tunnel that provides liquid communication between the first conductive liquid and second conductive liquid; c) applying an electrical potential between the first conductive liquid medium and the second conductive liquid medium to establish flow of electrical current through the tunnel of the portal protein; d) contacting the sensor with an analyte; and e) measuring a change in flow of electrical current through the tunnel of the portal protein indicative of translocation of the analyte, or a portion of the analyte, through the tunnel of the portal protein. The method can further include: f) correlating the measurements obtained in step e) to known standards to determine characteristics of the analyte.

A negative electrical potential can be applied across the membrane. The flow of electrical current through the tunnel of the portal protein can be at least about −200 mV, at least about −300 mV, at least about 400 mV, at least about −500 mV, at least about −600 mV, or at least about −750 mV.

The analyte can be a polynucleotide, a polypeptide, or a combination thereof.

Described herein is an apparatus for characterizing a target analyte. The apparatus can include at least one sensor having a thiol-silane surface and a nanopore, with a portal protein through the nanopore. The membrane is covalently bonded to the portal protein by a disulfide bond between a cysteine residue of the portal protein and the thiol-silane surface of the membrane. The apparatus can include a plurality of sensors arranged in an array.

Described herein is use of a sensor having a thiol-silane surface and a nanopore, with a portal protein through the nanopore. The membrane is covalently bonded to the portal protein by a disulfide bond between a cysteine residue of the portal protein and the thiol-silane surface of the membrane.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A shows the molecular surface of the G20c portal protein showing 1.8 nm constriction formed by tunnel loops of 12 subunits. Only 6 subunits are shown to visualize the central tunnel (vertical). FIG. 1B shows a schematic for disulfide bridge formation between cysteine residues of the CD/N G20c portal protein and the thiolated substrate. For clarity only 2 subunits of the portal protein are shown. FIG. 1C shows insertion of the CD/N G20c portal protein into the thiolated SiNₓ nanopore and formation of disulfide bridges between the protein and the pore wall ensures a highly stable hybrid nanopore. FIG. 1D shows 2D class averages calculated using cryo-EM images of CD/N mutant of the G20c portal protein. FIG. 1E shows functionalization of SiNₓ surface with MPTES.

FIG. 2A shows functionalization of SiNₓ surface with thia-silane through ring-opening click chemistry. FIG. 2B shows nanopore diameter change after silanization results in decreased ionic conductance. D′_{G}: nanopore diameter calculated from the nanopore conductance using the nanopore resistance model. D_{TEM}: nanopore diameter from TEM image taken after fabrication. The plot shows d_{TEM}−d′_{G} vs. d_{TEM} demonstrating the deviation of d′_{G} from d_{TEM} for both bare and silanized SiNₓ pores. The error bars show the values calculated with effective nanopore thickness ranging from 10-15 nm. FIG. 2C shows ionic current trace of a 7 nm bare SiNₓ nanopore shown in black and a 7 nm silanized SiNₓ nanopore shown in purple. The inset shows PSD of both pores at 100 mV. Traces were recorded at 250 kHz sampling frequency and lowpass filtered at 10 kHz. Buffer: 0.5 M NaCl, 20 mM HEPES, pH 7.5.

FIG. 3A shows an example of a successful hybrid nanopore formation through disulfide bridge formation between the portal protein and thiolated nanopore surface. The sudden drop in the ionic current indicates the protein insertion into the SiNₓ pore which is also stable at the reverse voltage. The schematic shows the direction of the applied force on the protein in each electric field. Traces were recorded at 100 kHz sampling frequency and lowpass filtered at 10 kHz. Buffer: 0.5 M NaCl, 20 mM HEPES, pH 7.5, 160 mM Cu(phenanthroline)₂. FIG. 3B shows an example of unsuccessful hybrid nanopore formation as indicated by ejection of the portal protein from the SiNₓ nanopore by a sudden increase in the ionic current at the reverse voltage. Traces were recorded at 250 kHz sampling frequency and lowpass filtered at 10 kHz. Buffer: 0.5 M NaCl, 20 mM HEPES, pH 7.5, 320 mM Cu(phenanthroline)₂. FIG. 3C shows a current-voltage curve of a 7-nm diameter silanized SiNₓ nanopore, before and after portal protein insertion. This graph shows the stability of our hybrid system up to 500 mV. FIG. 3D shows success rate for chemically bound hybrid nanopore formation with and without the use of a catalyst (catalytic reaction depicted above the plot). Unsuccessful hybrid pore formation is defined by portal ejection from the SiNₓ pore upon voltage reversal, as shown in FIG. 3B. FIG. 3E shows current-voltage curve of a 7-nm diameter silanized SiNₓ nanopore, before and after portal insertion.

FIG. 4A shows schematic of experimental setup. FIG. 4B shows 5-second current traces at −200 mV, −300 mV, and −400 mV demonstrate transient drops of the ionic current, characteristic signals of nanopore occlusion by DNA molecules (ssDNA final concentration: 6.25 μM). Traces were recorded at 100 kHz sampling frequency and lowpass filtered at 10 kHz. FIG. 4C shows three representative events at −200 mV, −300 mV, and −400 mV. FIG. 4D shows mean dwell time as a function of voltage showing that below −200 mV, ssDNA molecules only collide with the portal protein while at voltages higher than 200 mV DNA ssDNA molecules transport through the portal protein in the crown-to-clip direction as confirmed by the decrease in the dwell time as the applied voltage increases. Dwell time average values were calculated from the exponential fit to the distributions (FIG. 4G). The dashed line shows an exponential fit to the points. Data point at 380 mV is an outlier and was excluded in the fitting process. Buffer: 0.5 M NaCl, 20 mM HEPES, pH 7.5, 160 M Cu(phenanthroline)₂. FIG. 4E shows scatter plots of current blockage versus dwell time for events from −140 mV to −400 mV in 20 mV steps. At voltages higher than −300 mV a second population of events emerge shown with circles. FIG. 4F shows dwell time histogram of events from −140 mV to −400 mV in 20 mV steps. Lines show exponential fits to the histograms. Starting from −320 mV, as the second population of events start to emerge, double exponential function were fitted; however, there are not enough data points at −380 mV and −400 mV for double-exponential fitting. FIG. 4G shows mean dwell time as a function of voltage for second population of events shown in FIG. 4F. For −380 mV and −400 mV there were not enough data points to fit the distribution to the double-exponential function and deduce the second dwell time (FIG. 4E).

FIG. 5A shows schematic of experimental set-up. FIG. 5B shows 5-s current traces at 40 mV, 60 mV, 80 mV and 100 mV, demonstrating transient drops of the ionic current, characteristic signals of nanopore occlusion by DNA molecules (ssDNA concentration: 10 μM). Traces were recorded at 250 kHz sampling frequency and low-pass filtered at 10 kHz. FIG. 5C shows Scatter plots of current blockage versus dwell time for events from 40 mV to 100 mV in 20 mV steps. Buffer: 0.5 M NaCl, 20 mM HEPES, pH 7.5, 160 μM Cu(Phenanthroline)₂. FIG. 5D shows mean dwell time as a function of voltage showing transport of DNA molecules through the portal protein occurs at higher voltages than 80 mV. FIG. 5E shows normalized capture rate as a function of voltage shows increase in the capture rate by increasing voltage. Average values were calculated from the exponential fit to the distributions and error bars show weighed standard deviation.

FIG. 6A shows schematic of experimental set-up for motor protein-controlled DNA transport through a hybrid pore. FIG. 6B shows current traces at −200 mV, −300 mV, −400 mV, −500 mV, and −600 mV, demonstrating transient drops of the ionic current, which was attributed to DNA molecules ratcheting through the portal protein (traces were digitized using a sampling rate of 100 kHz after lowpass filtering at 10 kHz). To the right of each trace is a close-up view of the highlighted regions at each voltage. The traces were further lowpass filtered at 2 kHz. At 200 mV, the dashed lines highlight the stepwise change in the ionic current, which was attributed to DNA ratcheting motion. FIG. 6C shows scatter plots of current blockage versus dwell time at 300 mV and 400 mV to demonstrate a side-by-side comparison of DNA transport dynamics with and without ratcheting. Buffer: 0.5 M NaCl, 20 mM HEPES, pH 7.5, 320 M Cu(phenanthroline)₂, 75 mM KCl, 7 mM ATP, 7 mM MgCl₂. FIG. 6D shows examples of individual events for DNA feeding through the portal protein by the motor protein. FIG. 6E shows scatter plots of current blockage versus dwell time for events from −200 mV to −750 mV in 50 mV steps.

FIG. 7A shows a schematic of an experimental set-up. FIG. 7B shows current trace at −600 mV. The bottom trace shows the zoomed-in view of the highlighted events. FIG. 7C shows current trace at −650 mV. FIG. 7D shows current trace at −750 mV. The bottom traces show the close-up look of the highlighted regions. Traces were recorded at 100 kHz sampling frequency. Main traces were low-pass filtered at 10 kHz and the events were further filtered at 2 kHz. Buffer: 275 mM NaCl, 20 mM HEPES, pH 7.5, 320 mM KCl, 45 mM ATP, 45 mM MgCl₂.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines, and animals. The present disclosure is not limited by the illustrated ordering of acts or events, as some acts may occur in different orders and/or concurrently with other acts or events. Furthermore, not all illustrated acts, steps, or events are required to implement a methodology in accordance with the present disclosure. Many of the techniques and procedures described, or referenced herein, are well understood and commonly employed using conventional methodology by those skilled in the art.

Unless otherwise defined, all terms of art, notations, and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an,” and “the” should be understood to include plural reference unless the context clearly indicates otherwise.

Throughout this specification and the claims which follow, unless the context requires otherwise, the word “comprise,” and variations such as “comprises” and “comprising,” will be understood to imply the inclusion of, e.g., a stated integer or step or group of integers or steps, but not the exclusion of any other integer or step or group of integers or steps. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary the scope of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Sensors in accordance with an embodiment of the invention, including a portal protein supported within a membrane, combine the robust nature of membranes with the easily tunable and precise engineering of portal proteins. A lipid-free sensor comprises a water soluble and stable, modified portal protein of the Thermus thermophilus bacteriophage G20c, electrokinetically inserted into a nanopore in a membrane. The sensor is stable and easy to fabricate, and exhibits low peripheral leakage, allowing sensing and discrimination among different types of biomolecules.

The signal for sensing using the sensor of the disclosure can be either electrical or optical, the latter offering high-density parallelized readout from multiple adjacent pores. Embodiments include mechanisms to obtain a hybrid structure, to stabilize it, and to modify it so that different types of biomolecules can be sensed.

In embodiments, the sensor does not require any lipid support, which is typically fragile and not durable; it allows atomic-precision engineering to chemically define the pore sensor properties; and chemical methods of stabilizing the portal-to-solid-state interface are controlled by biomolecular engineering and materials science approaches. The sensor can, for example, provide the advantages of: rapid and stable insertion of a protein into a solid-state nanopore; mutations of the protein can be used for sensing improvement; and translocation of biopolymers (such as nucleic acids and polypeptides) through the hybrid sensor can be performed for sensing applications. Example potential merits of such a device are in applications that include: 1) high-resolution mapping of DNA, RNA sequencing, DNA sequencing; 2) protein identification, protein conformational change monitoring; 3) polypeptide sequencing; 4) small-molecule detection, biomolecular complex detection, and enzyme-ligand binding. The broad range of uses could potentially impact many areas of the human health, biotechnology and agri-food sectors.

The advent of single-molecule detection is having an unparalleled impact on the speed with which structural and dynamic aspects of molecules can be probed. In this regard, nanopore sensors have shown much promise as electrical and electro-optical sensors and several nanopore-based systems are now being adopted as primary tools for DNA and RNA sequencing.

Despite recent progress, identification and quantification of molecular species in solution requires a reproducible nanopore platform that affords physical stability, structural precision, and often, a spatially-defined pore position (for example, in electro-optical sensing). While synthetic nanopores fabricated in solid-state (SS) membranes offer physical robustness, pore-to-pore variability often limits the reproducibility of experiments, necessitating additional control checks and validation. On the contrary, portal protein channels embedded in organic thin membranes (e.g., a lipid-bilayer) offer the highest reproducibility due to the precise folding and repetitive nature of the constituting multi-subunit protein oligomers, but their supporting membrane is typically less chemically and physically robust, and further, the pore position is not well-defined due to in-plane diffusion of the portal protein channel. Hybrid nanopore devices, in which channel-containing proteins are embedded in larger pores made in a SS matrix, have been proposed as a strategic solution for combining the benefits—while overcoming the limitations—of existing nanopores. Although initial experiments based on inserting pore-containing proteins with lipophilic regions into a SS pore looked promising, challenges in inserting such proteins into a SS pore and in controlling the protein orientation have remained major obstacles in the applicability of hybrid nanopores to nanotechnology.

In addition, it should be noted that monomer protein units of proteins taught herein can be assembled to form the full portal protein that functions to form the stable insertion fit within a solid-state pore opening that is taught herein. For example, the portal protein of the Thermus thermophilus bacteriophage G20c forms a dodecameric structure, made of 12 monomer protein units, which together assemble to form the full “plug” protein that forms a stable insertion fit within the solid-state pore opening. Thus, a “hydrophilic protein channel,” as used herein, can include more than one monomer of a protein, such as 12 monomer protein units assembled together to form the hydrophilic protein channel through the dodecameric combined protein structure assembled from the monomers of the protein.

### EXEMPLIFICATION

### Materials and Methods

Protein Cloning, Expression, and Purification: CD/N mutant portal protein (SEQ ID NO: 3) was expressed and purified as explained previously.³⁹ In summary, this mutant was expressed in Escherichia coli shuffle cells after reaching OD600=0.8 and induction with 0.5 mM isopropyl β-D-1-thiogalactopyranoside (IPTG) at 30° C. overnight. Afterward, the cell pellets were resuspended in 1 M NaCl, 50 mM Tris pH 8, 10 mM imidazole, 2 mM DTT, and kept at −80° C. until purification. For purification, the cells were thawed, lysed by sonication after addition of 10 mg·ml⁻¹ lysozyme, protease inhibitor tablets (Thermo Scientific, A32963), and further clarified by centrifugation at 12 k rpm for 55 minutes. Protein purification included 6 steps: 1. Purification by Immobilized Metal Affinity Chromatography (IMAC; 5 mL HiTrap FF Crude, GE Healthcare), 2. Buffer exchange to 0.5 M NaCl, 50 mM Tris pH 8, 50 mM potassium Glutamate, 1 mM DTT using a desalting column (HiPrep 26/10; GE Healthcare), 3. Overnight cleavage of the histidine affinity tag using 3C protease, 4. buffer exchange into 1 M NaCl, 50 mM Tris pH 8, 10 mM Imidazole, 2 mM DTT using the same desalting column, 5. Purification by IMAC for the second time to remove histidine-tagged proteins, 6. Purification by Size Exclusion Chromatography (SEC; Superose 18/300; GE Healthcare). The purified proteins were flash-frozen and kept at −80° C. until use.

Silicon Nitride Membrane Fabrication: 50-nm-thick free-standing SiNₓ membranes were fabricated at the center of 5 mm×5 mm silicon chips. In summary, 50-nm-thick, high-stress (250 Mpa) SiNₓ film was deposited on a 2 μm-thick SiO₂ layer/300 μm-thick silicon wafers. Photolithography (Karl Suss MA6 mask aligner) and several wet and dry etching steps were used to pattern the final 20 μm×20 μm SiNₓ membranes. 3 μm circular areas on the membrane (known as thin regions) were locally thinned to 25-30 nm using Technics Micro-RIE Series 800 for SF6 plasma etching (200 mTorr, 50 W, 40 s) and further confirmed by AFM.

Solid-State Nanopore Chemical Modification: 7-8 nm nanopores were fabricated through the thin region of the SiNₓ membranes using a transmission electron microscope (TEM) beam. Each chip containing the membrane and fabricated pore was thoroughly cleaned first by immersing in hot piranha solution (1:2 H₂O₂:H₂SO₄) for 30 minutes and hot water for 30 minutes for any organic residue and contamination to be removed and the surface to be rendered hydrophilic and hydroxylated (piranha solution was always used freshly prepared in glassware while kept inside a fume hood). Afterward, chips were dried entirely using an N₂ gun and baked at 150° C. for 3 minutes. The silanization protocol was adapted from the previous work by Kim et al.⁴⁵ Each chip was placed into a 1.5 ml Eppendorf vial and moved into the glovebox with running N₂ gas. 400 μl extra dry dichloromethane (Acros Organics) and 100 μl 2,2-dimethoxy-1-thia-2-silacyclopentane (Gelest, Inc.) were added to each vial and left for the silanization to complete for 1-3 hours. To remove the chemisorbed molecules on the surface, each chip was immersed in 5 vials of DCM and gently agitated. Each chip was kept in anhydrous ethanol (Acros Organics) until use and was dried before loading on the fluidic cell. Silicone elastomer was used to seal the chip edges. Ethanol was used to facilitate wetting of the silanized pores.

Nanopore Experiment Data Acquisition and Analysis: Axopatch 200B amplifier at either 100 kHz or 250 kHz sampling rate was used for single-channel measurements. After wetting the silanized pores using ethanol and getting a stable current, portal protein with a final concentration of ˜20 μg/μl was added to the trans chamber and Cu(phenanthroline)₂, with a final concentration of 160-320 μM, was added to both chambers. After portal protein insertion into the SiNₓ nanopores, detected by a sudden drop in the ionic current, voltage was reversed to check the protein's stability. If the protein was stable at both voltages and the hybrid nanopore was successfully formed, the desired analyte was added to the trans or cis chamber. Oxford Nanopore rapid sequencing kit (SQK-RAD004) was used for motor protein/DNA experiments. Data processing was done using OpenNanopore software and Pythion software and further analyzed using Igor Pro software. For dwell time distributions, the first two bins representing events <150 μs were excluded in the fitting process due to the underrepresentation of events close to the 10 kHz filtering threshold.

Supporting Information: 2D class averages of CD/N mutant of the G20c portal protein, schematics of functionalization of SiNₓ surface with MPTES, further example of hybrid nanopore I-V curve, scatter plots of current blockage versus dwell time, dwell time histograms, and mean dwell time as a function of voltage of DNA molecules transporting through the hybrid nanopore in the crown-to-clip direction, ssDNA molecules transport and its dynamics in the clip-to-crown direction, further examples of events and scatter plots of current blockage versus dwell time for DNA molecules feeding through the portal protein by the motor protein, and ssDNA ratcheting events through a chemically modified SiNₓ nanopore.

### Results and Discussion

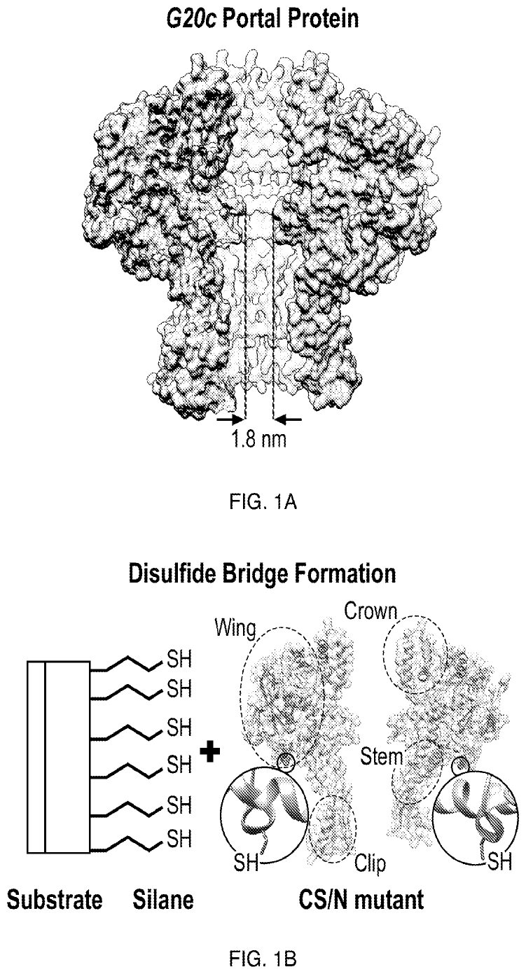

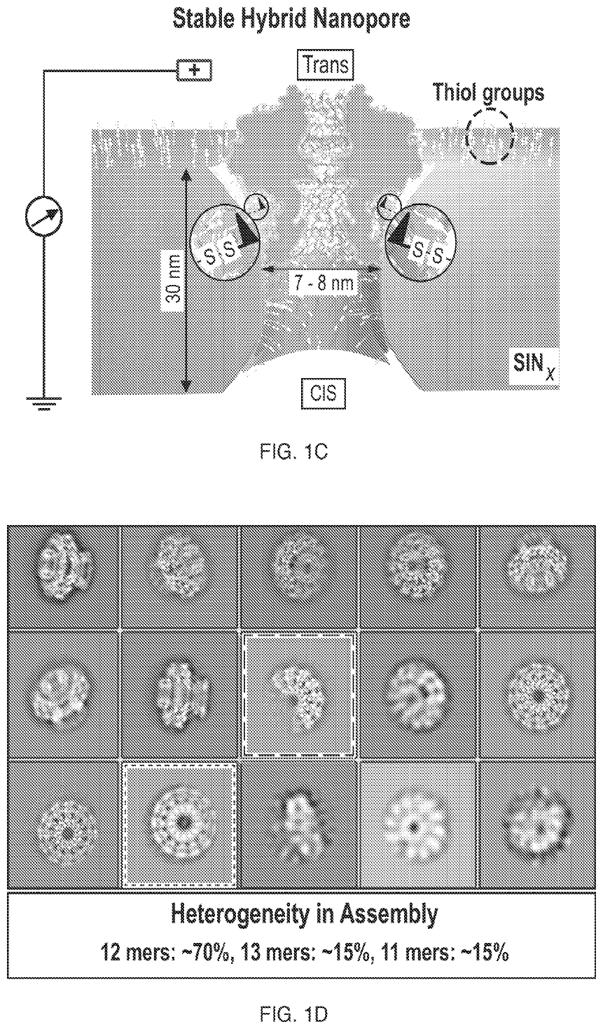

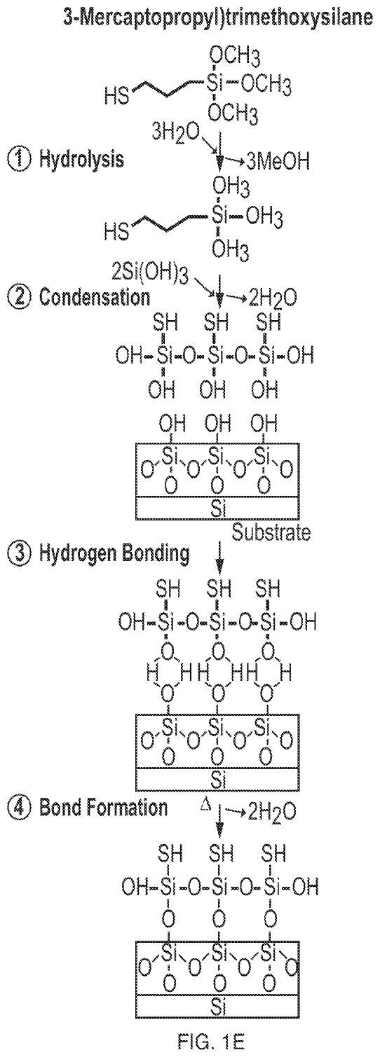

G20c portal protein (SEQ ID NO: 1) from the thermostable bacteriophage G20c oligomerizes to form a dodecameric assembly with an internal channel containing 1.8 nm constriction, as shown in FIG. 1A. In common with the wild-type protein, its CD/N mutant (SEQ ID NO: 3), where four aspartic acid residues (D) were substituted by asparagine (N) to alter the internal surface charge for enhanced sensing of negatively charged molecules,³⁹ in addition to substitution of an externally facing leucine residue to cysteine for chemical labeling,⁴⁰ also forms 12-mers (FIG. 1D). In contrast to the most successfully implemented proteins as nanopore sensors that assemble to form channels upon insertion into the lipid bilayer or synthetic polymer membranes, the hydrophilic G20c portal protein is water-soluble and fully assembles in an aqueous solution. So far, efforts have resulted in two distinct methods to utilize this protein as a nanopore sensor: (1) insertion into the lipid bilayer through a hydrophobic conjugate⁴⁰ and (2) corking into solid-state nanopore to form a lipid-free hybrid nanopore.³⁹ However, none of these methods have been efficient enough to deliver high-yield, full-range sensing capability in various experimental conditions, particularly failing at a high applied voltage.

Building upon previous work, the present disclosure exploits the cysteine residues residing under the wing and near the cap of the protein to form disulfide bridges with a thiolated solid-state nanopore, as shown in FIG. 1B. The ideal positioning of the cysteine mutation allows site-specific, reproducible, and uniformly oriented protein immobilization within the thiolated SiNₓ nanopore while preserving its conformation and activity. The dominant protein orientation within the SiNₓ nanopore, shown in FIG. 1C, is predicted to be required for the protein to cross-link to the chemically-modified substrate. Given this specific geometric point of the cysteine modification in the protein, formation of an undesirable disulfide bridge between the protein and the planar surface of the SiNₓ membrane is not feasible.

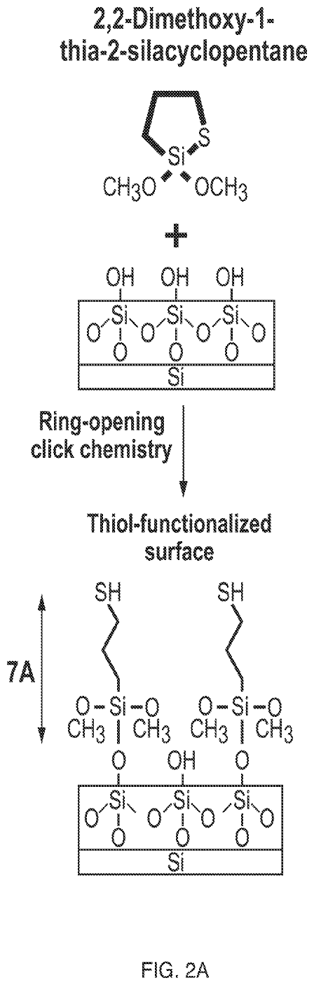

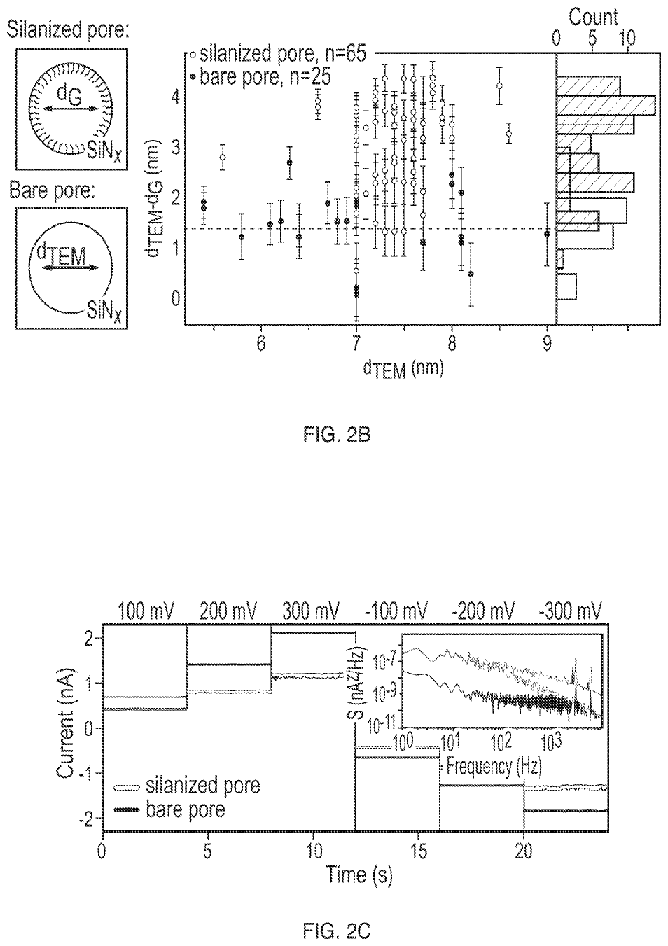

In order to chemically modify the SiNₓ membrane, silane coupling agents that form durable self-assembled monolayers (SAMs) through covalent bonding to SiOₓ-rich surfaces were used. SiNₓ is also amenable to silanization because it contains a silicon-rich, nitrogen-depleted area on its surface which is terminated by oxide and hydroxide groups.⁴¹ This native oxide layer is beneficial for direct chemical modification of SiNₓ nanopores with silanes.⁴²⁻⁴⁴ For purposes of the present disclosure, thiol functionality is imparted to the surface by treating the SiNₓ membrane with 2,2-dimethoxy-1-thia-2-silacyclopentane (thia-silane). Thia-silane reacts rapidly with the surface through ring-opening click reaction driven by the difference in bond energies and relief of the ring strain. This reaction is simple, fast, high-yield, and occurs via cleavage of Si—S bond by hydroxyl groups as previously reported.⁴⁵⁻⁴⁷ The ring structure of the thia-silane protects the sulfhydryl group from forming disulfide bonds prior to SAM formation (FIG. 2A). While other thiol-functionalized silanes such as 3-(mercaptopropyl)trimethoxysilane (MPTES) can be used,⁴⁸ the deposition on the substrate was found to be more prone to form multi-layers on the surface (FIG. 1E), which presumably occurs via several steps of hydrolysis and condensation reactions. To chemically modify the SiNₓ nanopores, 7-8 nm diameter pores were fabricated through a 30-nm thick SiNₓ membrane with a focused transmission electron microscope (TEM) beam and thoroughly cleaned with piranha solution for any organic residue and contamination to be removed and the surface to be rendered hydrophilic and hydroxylated. Afterward, the SiNₓ membranes were incubated with silane solution for 1-3 hours. Silanized pores were rinsed with the solvent in several steps and assembled in a fluidic cell with two 70 μl volume chambers across the chip for trans-pore ion current measurements. Ion transport through a nanopore could be related to the pore geometry using a pore conductance model that takes into account pore dimensions and an access resistance term. Thia-silane molecules with an extended length of ˜7 Å reduce the pore diameter and increase its overall thickness by approximately 1.4 nm. Therefore, silanized pores are expected to have less conductance than expected from their TEM-measured diameter (d_{TEM}), as shown in FIG. 2B.

Next, the pore resistance model was used to calculate the bare and silanized SiNₓ pore diameters from the ionic conductance after single-channel measurement (d′_{G}) and compare it to their d_{TEM}. FIG. 2B shows a systematic deviation of d′_{G} from d_{TEM} for both bare and silanized SiNₓ pores. While for the bare SiNₓ pores, the deviation is 1.51±0.63 nm, for silanized pores we find a deviation of 3.1±0.9 nm. While deviations for the bare pore are significant, they appear to be systematic, pointing to a systematic error between TEM-imaged pores and experimental measurements. However, the difference between unmodified and chemically-modified pores is consistent, with a 1.6 nm mean decrease in diameter after coating, which supports the interpretation that a successful coating was achieved. FIG. 2C shows current traces of two 7-nm SiNₓ pores, one silanized (open trace) and one bare (dark trace). As expected, the silanized pore has less ionic conductance yet is stable and has larger low-frequency noise, as shown by the power spectral density (PSD) plots at 100 mV (inset). Finally, in accordance with previous studies,^{49,50} a common outcome of chemical modification of a pore is that molecular fluctuations, formation of transient hydrophobic pockets, and charge fluctuations, all cause increases in 1/f noise.

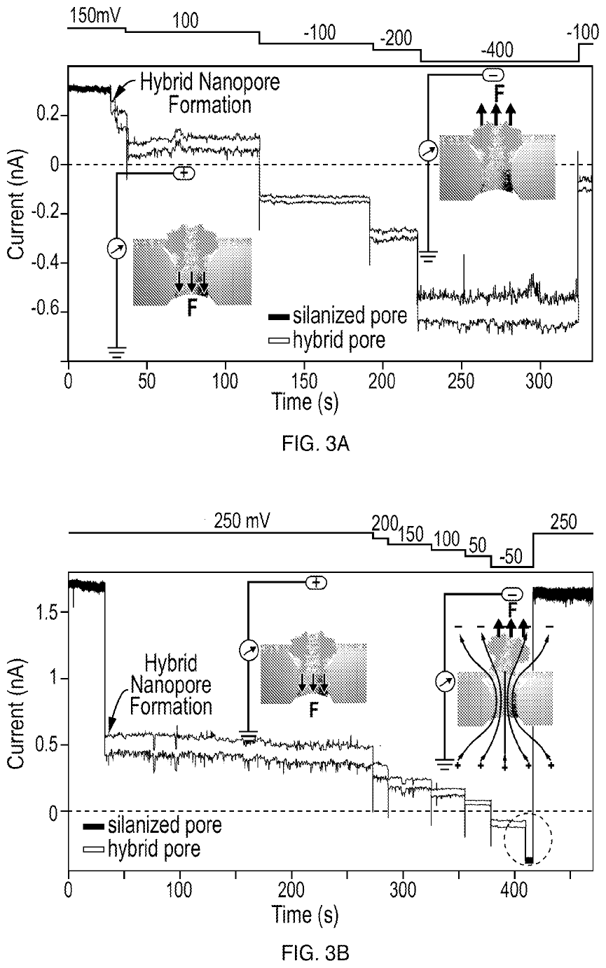

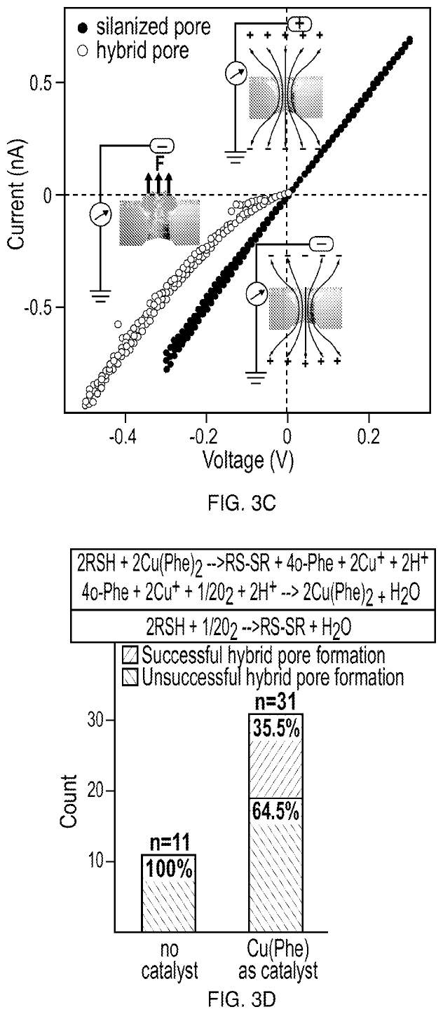

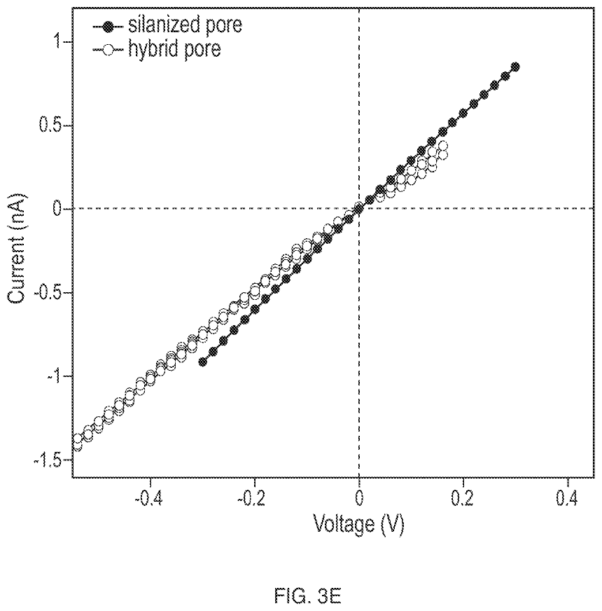

As shown in Cressiot et al.,³⁹ successful portal protein insertion and corking into a SiNₓ nanopore with desired orientation required maintaining a voltage bias across the membrane to electrokinetically drive the protein to the SiNₓ nanopore whose geometry is commensurate with the protein's structure and size. The charge bipolarity of the portal protein's external surface⁴⁰ prompts its orientation in the direction of the electric field, but the overall net force on the portal is such that reversing the bias results in ejection of the portal from the pore, limiting the applicability of our hybrid system at various applied voltages. The present disclosure implements the exact mechanism for the formation of the hybrid nanopore system along with chemically fixing the protein to the SiNₓ nanopore to inhibit its uncorking in reverse voltage and attain permanent immobilization of the portal protein within the support membrane. As shown in FIG. 3A, insertion of the protein into the silanized SiNₓ nanopore is observed by a drop in the ionic current. Successful disulfide bridge formation between the portal protein and the thiol groups on the pore walls was confirmed by reversing the voltage bias to −400 mV, which typically did not result in ejection of the portal protein from the SiNₓ nanopore. This indicates a stable hybrid nanopore with applicability in both voltage biases. An unsuccessful hybrid nanopore formation is easily identified as a sudden increase in the ionic current to an “open pore” conductance level when the voltage is reversed, as shown in FIG. 3B. FIG. 3C shows a current-voltage curve of a 7 nm silanized SiNₓ pore before and after hybrid nanopore formation. As seen in the current-voltage curve, the hybrid nanopore is stable at high voltages (up to 500 mV shown here). Negative voltages are shown only because in the positive voltage direction the portal is pushed into the pore, and moreover, in this polarity DNA cannot enter from the crown direction (FIG. 3E shows an IV curve for another hybrid pore before and after portal tethering). However, a hallmark of successful disulfide bridge formation between portal protein and silanized SiNₓ pore is the ability to apply large negative voltages to the hybrid pore without portal dissociation. This covalent linkage formation is mediated by the catalyst Cu(phenanthroline)₂, which facilitates the thiol oxidation through a mechanism previously reported in Kobashi et al.⁵¹ (shown in FIG. 3D). Successful disulfide hybrid pore disulfide bridge formation in the absence of this catalyst was not observed. Moreover, the arbitrary geometry of the fabricated SiNₓ pores and the deviation of their diameters, as shown in FIG. 2B, may render the geometry of some pores unfit for successful portal protein insertion or disulfide bridge formation.

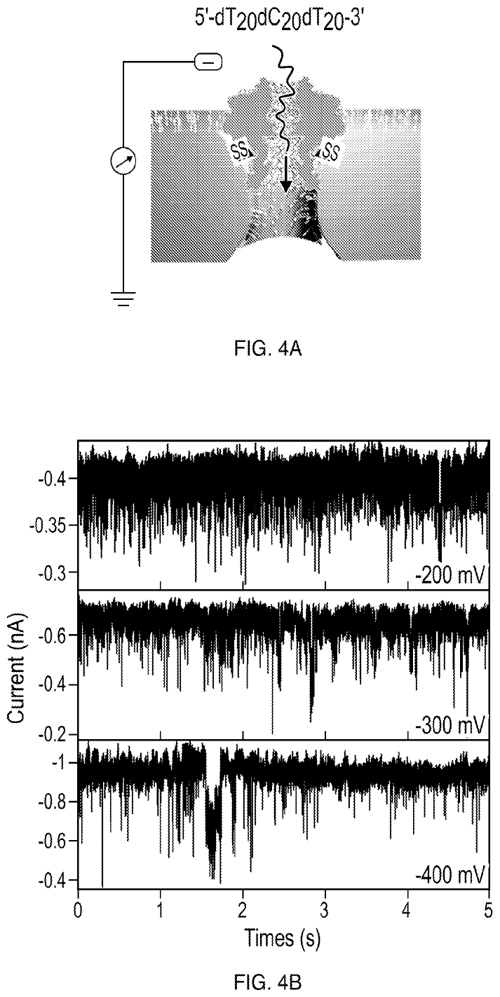

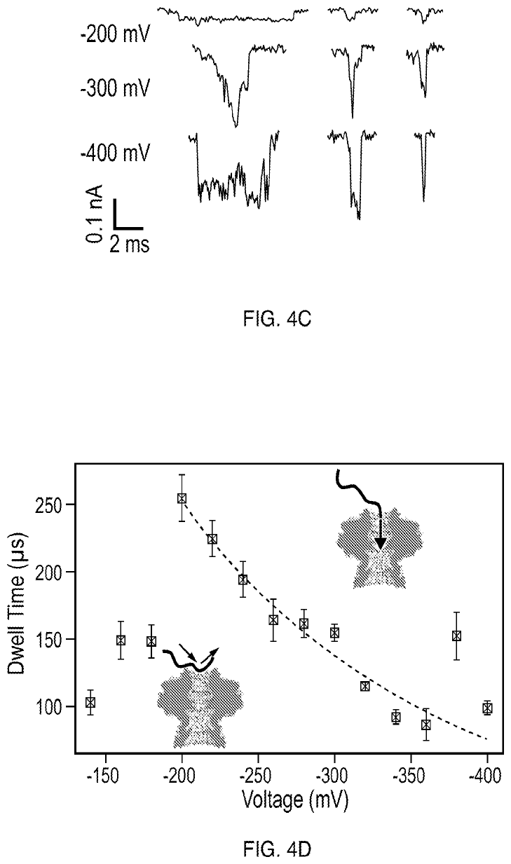

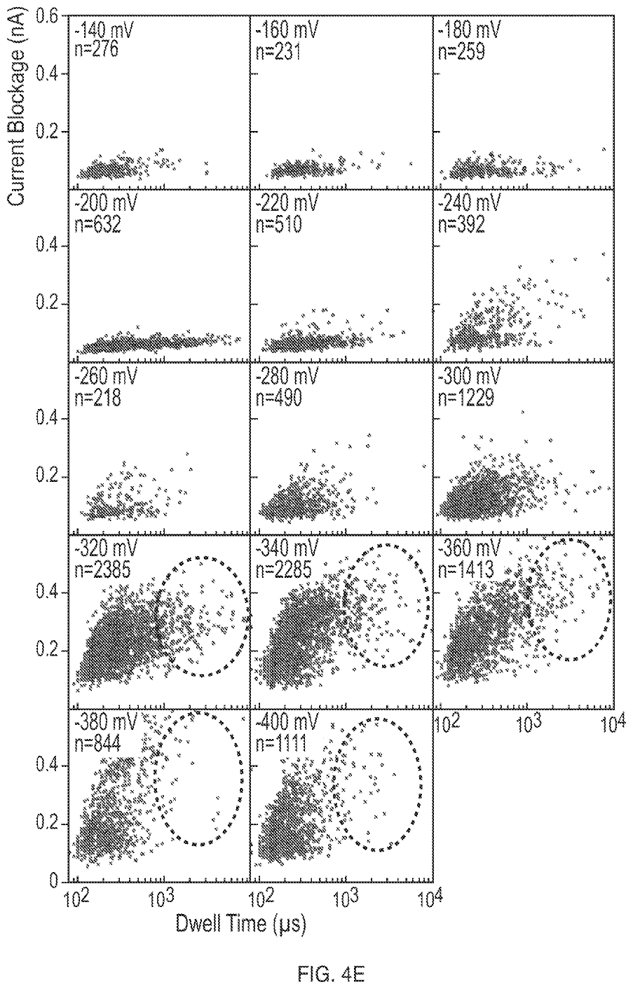

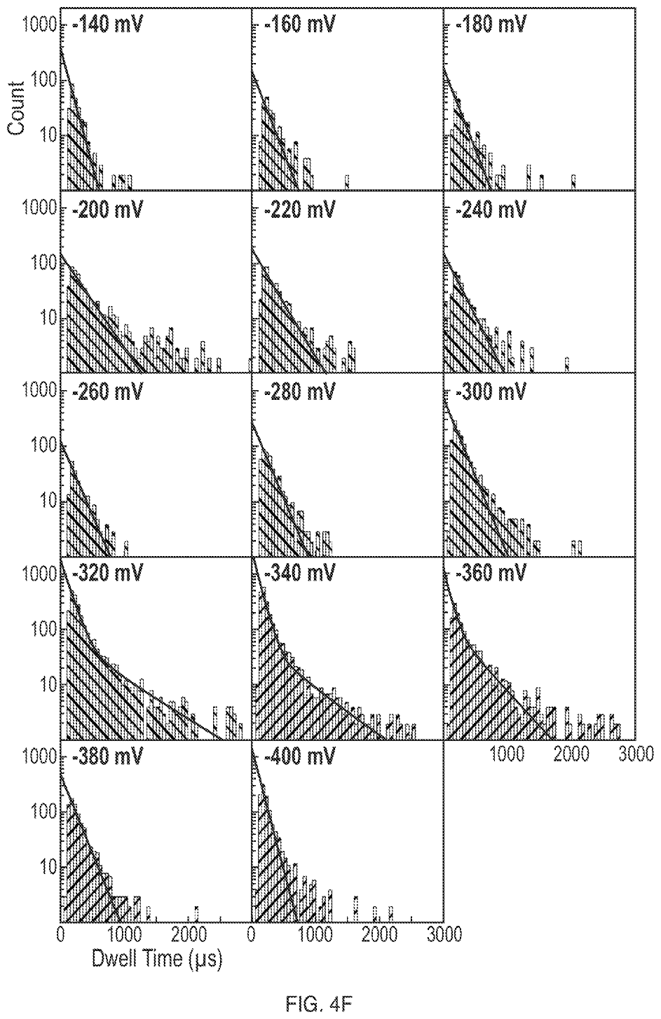

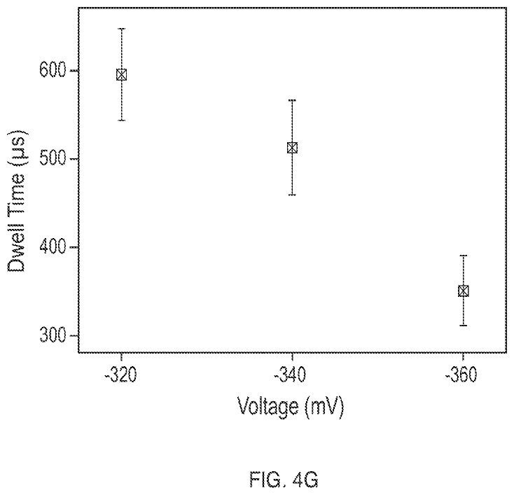

Next, the stable hybrid nanopore system's capability for sensing biomolecules through studying ssDNA translocation in both crown-to-clip and clip-to-crown directions was tested. Previous studies only probed biomolecule translocation in the clip-to-crown direction due to the corked portal hybrid pore's instability upon reversal of voltage polarity.³⁹ FIG. 4A shows a schematic of the experiment where negative voltage is applied to the trans chamber to facilitate ssDNA translocation in the crown-to-clip direction. FIG. 4B shows 5-second current traces of the hybrid nanopore at −200 mV, −300 mV, and −400 mV, where transient drops seen in the ionic current indicate temporary occlusions of the nanopore by the ssDNA molecules. FIG. 4C shows a close-up look at three representative events for each voltage. To confirm ssDNA molecules transport through the hybrid nanopore in the crown-to-clip direction, an analysis of the event characteristics is required. FIG. 4E shows scatter plots of ionic current blockage versus dwell time from −140 mV to −400 mV in 20 mV steps. The plots show that at voltages <220 mV, the events are primarily low-level current blockades with spread-out dwell times indicating collision of DNA molecules with the portal protein and failed translocation. While not serving as concrete proof, this behavior is consistent with a recent structural study which indicated the portal may serve as a one-way valve preventing dsDNA leakage from capsids during virus assembly.⁵² The mechanism involves “loop-in” conformation activated during transport in the crown-to-clip direction, when portal's crown adjustment triggered by DNA forces the internal tunnel loops in the pore lumen to extend towards DNA and prevent its translocation along the tunnel. The observed behavior in the low-voltage regime is also consistent and a former study on membrane-embedded portal with a similar portal mutant which showed that β-cyclodextrin transport does not occur in the crown-to-clip direction for the voltage range studied (<140 mV).⁴⁰

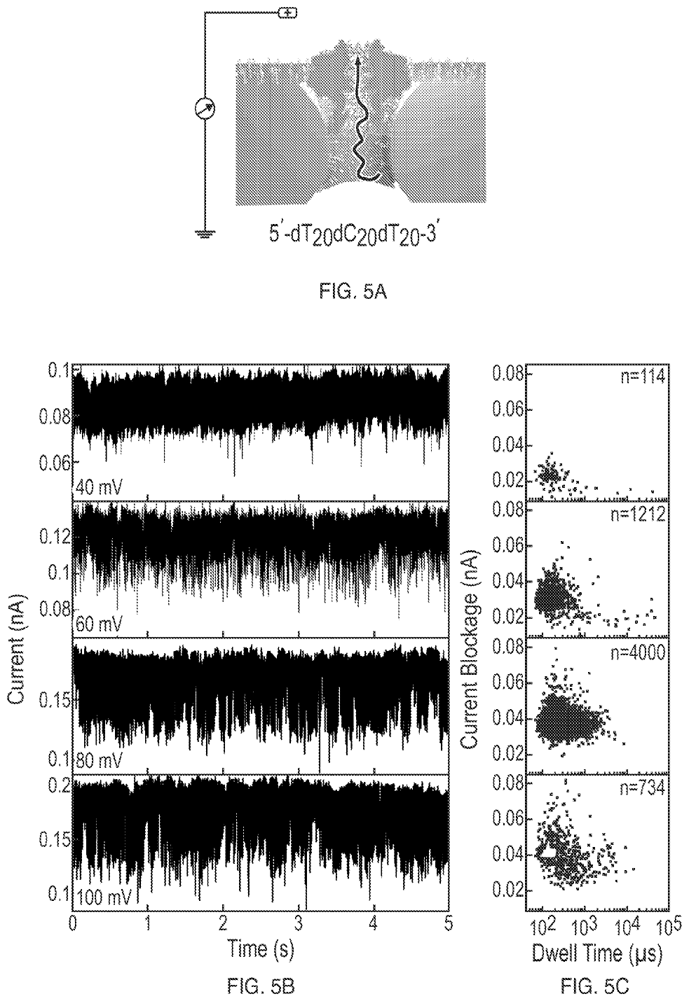

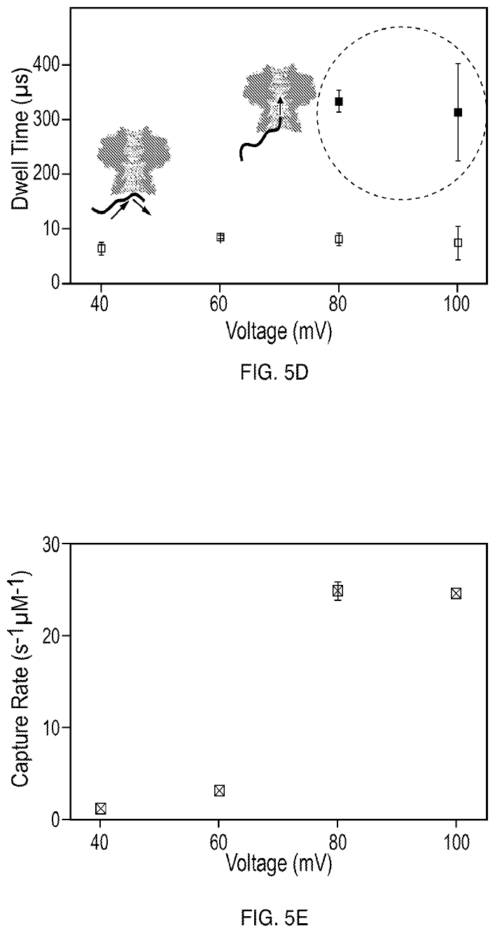

In contrast to the low voltage behavior, at voltages higher than 220 mV the scatter plots reveal a new population of events with deeper blockades and dwell times that decrease with increasing voltage. To study the dwell time change as a function of voltage, exponential functions were fitted to the dwell time distribution shown in FIG. 4F. FIG. 4D shows that from 140 to 220 mV, the mean dwell time of events increases with voltage, while from 220 to 400 mV, dwell time decreases with voltage. This suggests that below a certain voltage threshold (220 mV here) ssDNA molecules only collide with the portal protein, while above the threshold ssDNA molecules start to fully transport through the pore in the crown-to-clip direction. At voltages above ˜320 mV, a new (minor) population of slow events emerges, which was attributed to ssDNA/protein interaction or sticking (not shown in FIG. 4D, see 4G). The dynamics of ssDNA translocation in the clip-to-crown direction were studied by applying a positive voltage to the trans chamber and adding DNA to the cis chamber (FIG. 5A). FIG. 5B shows 5-second current traces of the hybrid nanopore after adding ssDNA at 40 mV, 60 mV, 80 mV, and 100 mV. Analysis of the events (FIGS. 5C and 5D) show that at all voltages, there is a population of fast events that does not depend on voltage which is characteristic of ssDNA collision with the portal protein. At 80 mV and 100 mV, the second population of events with higher dwell time emerges, which was attributed to DNA transport through the portal protein in the clip-to-crown direction. The results demonstrate that there is less threshold for ssDNA molecules to transport in the clip-to-crown direction than in the crown-to-clip direction, probably because the structure of the portal entry in the crown direction contains more void volume that accommodates various ssDNA configurations, which disfavors threading and translocation. Analysis of the capture rate as a function of voltage (FIG. 5E) depicts an increase in the rate with increasing voltage.

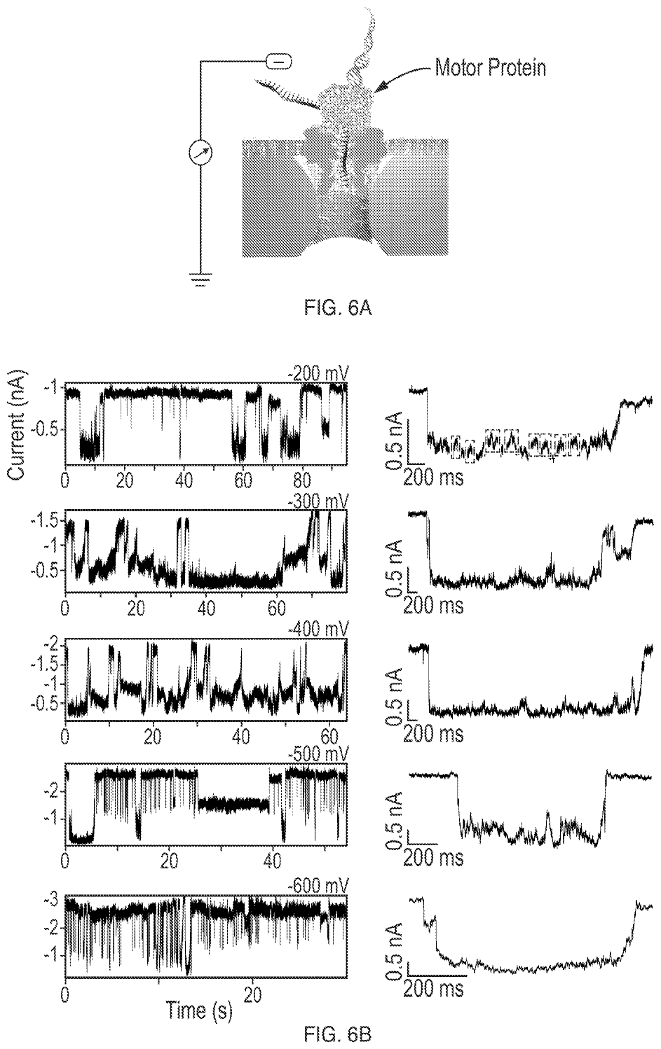

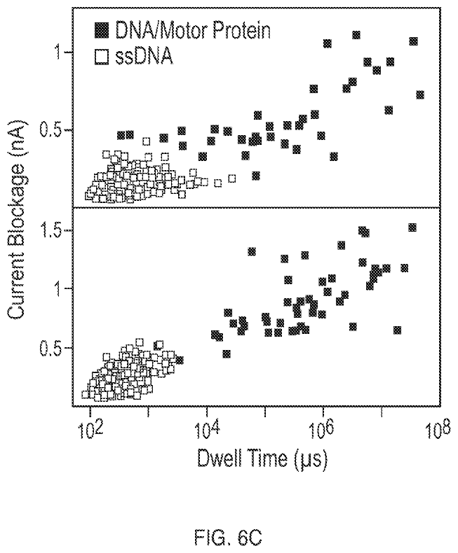

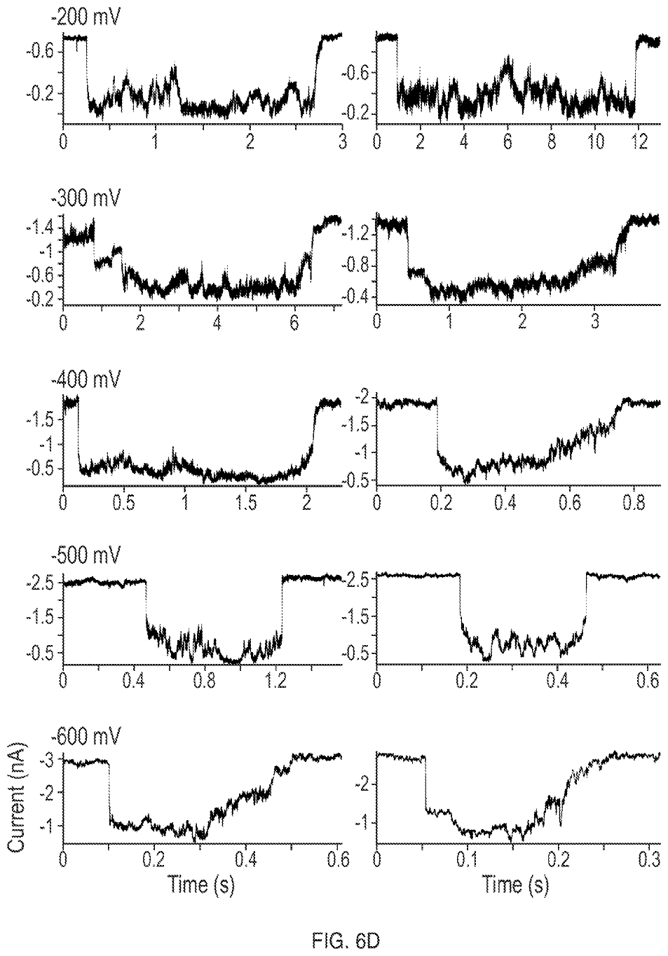

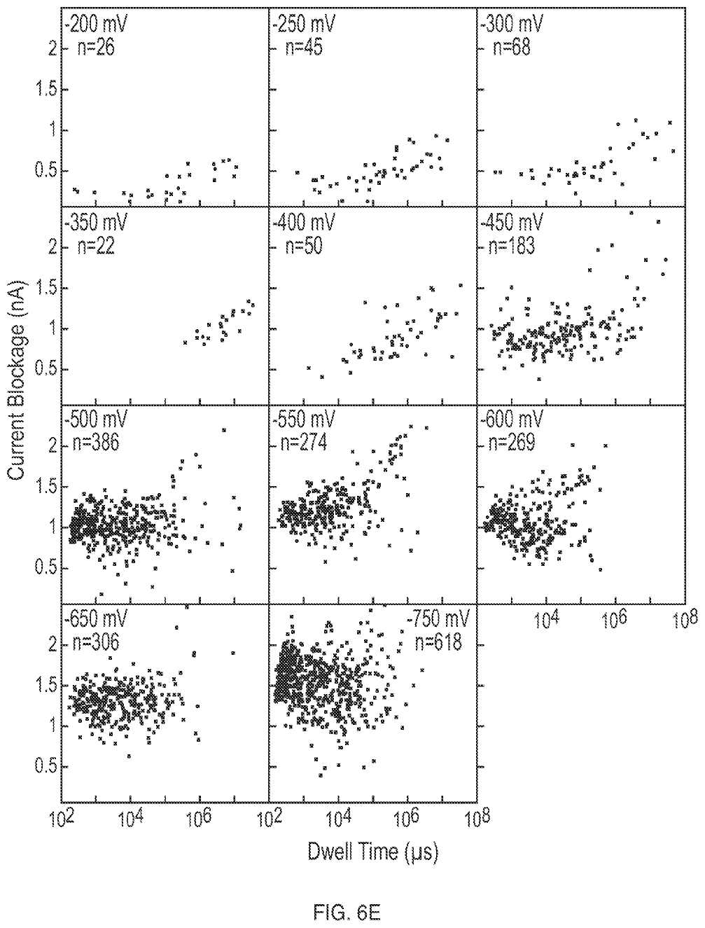

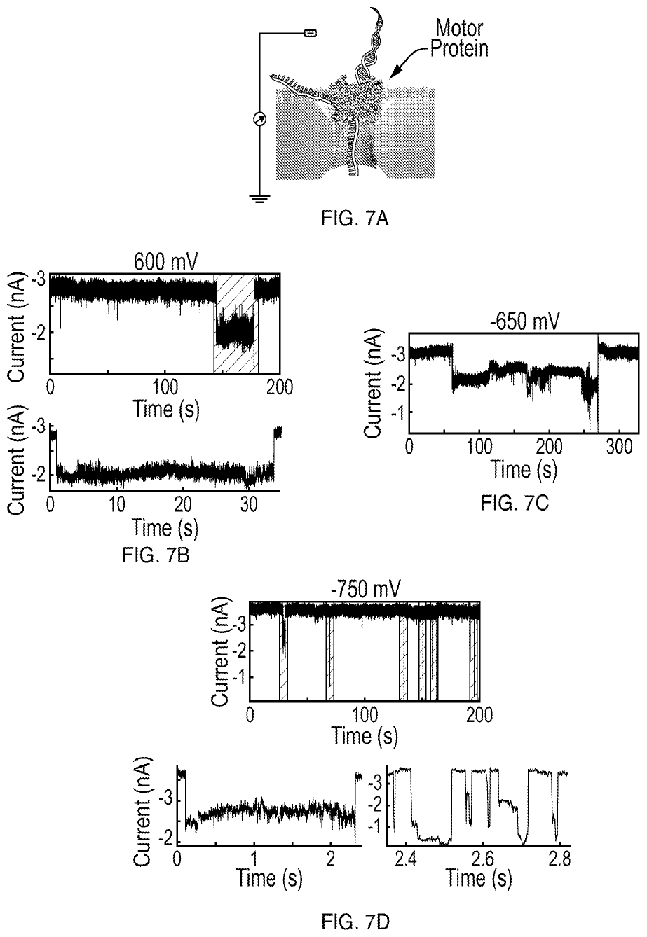

To further test the hybrid system for high-voltage sensing and sequencing applications, DNA ratcheting through the hybrid nanopore using an Oxford Nanopore Technologies sequencing chemistry was studied. FIG. 6A shows a schematic of the experiment where a motor protein bound to its DNA molecule rests on the portal protein and ratchets its DNA through the pore. The experiment was carried out at voltages from −200 mV to −750 mV to demonstrate the activity of our hybrid system at high voltage. FIG. 6B shows current traces from −200 mV to −600 mV. On the right side, a close-up view of the highlighted regions is shown. At 200 mV, distinct features correlated with the stepwise motion of the DNA through the portal protein are visible, while at higher voltages these features become less frequent (FIG. 6D shows more examples of events). FIG. 6C demonstrates scatter plots of current blockage vs. dwell time at 300 mV and 400 mV for both freely transporting ssDNA and motor protein-bound DNA molecules. This side-by-side comparison shows that the motor protein feeds DNA into the pore ˜5 orders of magnitude slower. Moreover, as indicated by current traces at different voltages and dwell time decrease as a function of voltage, DNA strips off from the motor protein at high voltages (>450 mV) due to the high electrostatic force intervening with the ratcheting process (FIG. 6E). Therefore, although the hybrid nanopore system shows stable activity up to 750 mV, the motor protein is not optimized for use at high voltages. In a control experiment using motor protein/DNA with silanized SiNₓ nanopore (FIG. 6E) at high voltage (>600 mV), signals are less frequent and completely distinct from equivalent signals in the hybrid nanopore, which confirms that these signals are indeed due to DNA ratcheting through the portal protein.

TABLE 1
Summary of sequences

| SEQ ID NO: | Description |
|---|---|
| 1 | Wild Type G20c protein sequence |
| 2 | Wild Type G20c DNA sequence |
| 3 | G20c CD/N mutant protein sequence |
| 4 | G20c CD/N mutant DNA sequence |

As used herein, the term “sequence identity,” refers to the extent to which two sequences have the same residues at the same positions when the sequences are aligned to achieve a maximal level of identity, expressed as a percentage. For sequence alignment and comparison, typically one sequence is designated as a reference sequence, to which a test sequences are compared. Sequence identity between reference and test sequences is expressed as a percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide residue at 70% of the same positions over the entire length of the reference sequence.

Alignment of sequences for comparison to achieve maximal levels of identity can be readily performed by a person of ordinary skill in the art using an appropriate alignment method or algorithm. In some instances, alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology). In some embodiments, codon-optimized sequences for efficient expression in different cells, tissues, and/or organisms reflect the pattern of codon usage in such cells, tissues, and/or organisms containing conservative (or non-conservative) amino acid substitutions that do not adversely affect normal activity.

### REFERENCES

1. Akeson, M.; Branton, D.; Kasianowicz, J. J.; Brandin, E.; Deamer, D. W., Microsecond Time-Scale Discrimination among Polycytidylic Acid, Polyadenylic Acid, and Polyuridylic Acid as Homopolymers or as Segments within Single RNA Molecules. Biophysical journal 1999, 77 (6), 3227-3233.
2. Fologea, D.; Ledden, B.; McNabb, D. S.; Li, J., Electrical Characterization of Protein Molecules by a Solid-State Nanopore. Applied Physics Letters 2007, 91 (5), 053901.
3. McNally, B.; Wanunu, M.; Meller, A., Electromechanical Unzipping of Individual DNA Molecules Using Synthetic Sub-2 nm Pores. Nano Letters 2008, 8 (10), 3418-3422.
4. Wang, H.; Dunning, J. E.; Huang, A. P. H.; Nyamwanda, J. A.; Branton, D., DNA Heterogeneity and Phosphorylation Unveiled by Single-Molecule Electrophoresis. Proceedings of the National Academy of Sciences of the United States of America 2004, 101 (37), 13472.
5. Smeets, R. M. M.; Kowalczyk, S. W.; Hall, A. R.; Dekker, N. H.; Dekker, C., Translocation of RecA-Coated Double-Stranded DNA through Solid-State Nanopores. Nano Letters 2009, 9 (9), 3089-3095.
6. Wanunu, M.; Dadosh, T.; Ray, V.; Jin, J.; McReynolds, L.; Drndic, M., Rapid Electronic Detection of Probe-Specific microRNAs Using Thin Nanopore Sensors. Nat. Nanotechnol. 2010, 5 (11), 807-14.
7. Pastoriza-Gallego, M.; Rabah, L.; Gibrat, G.; Thiebot, B.; van der Goot, F. G.; Auvray, L.; Betton, J.-M.; Pelta, J., Dynamics of Unfolded Protein Transport through an Aerolysin Pore. Journal of the American Chemical Society 2011, 133 (9), 2923-2931.
8. Yang, J.; Wang, Y.-Q.; Li, M.-Y.; Ying, Y.-L.; Long, Y.-T., Direct Sensing of Single Native Rna with a Single-Biomolecule Interface of Aerolysin Nanopore. Langmuir 2018, 34 (49), 14940-14945.
9. Cao, C.; Ying, Y.-L.; Hu, Z.-L.; Liao, D.-F.; Tian, H.; Long, Y.-T., Discrimination of Oligonucleotides of Different Lengths with a Wild-Type Aerolysin Nanopore. Nature Nanotechnology 2016, 11 (8), 713-718.
10. Singer, A.; Rapireddy, S.; Ly, D. H.; Meller, A., Electronic Barcoding of a Viral Gene at the Single-Molecule Level. Nano Letters 2012, 12 (3), 1722-1728.
11. Gu, L.-Q.; Wanunu, M.; Wang, M. X.; McReynolds, L.; Wang, Y., Detection of miRNAs with a Nanopore Single-Molecule Counter. Expert Review of Molecular Diagnostics 2012, 12 (6), 573-584.
12. Zahid, O. K.; Zhao, B. S.; He, C.; Hall, A. R., Quantifying Mammalian Genomic DNA Hydroxymethylcytosine Content Using Solid-State Nanopores. Scientific Reports 2016, 6 (1), 29565.
13. Kong, J.; Bell, N. A. W.; Keyser, U. F., Quantifying Nanomolar Protein Concentrations Using Designed DNA Carriers and Solid-State Nanopores. Nano Letters 2016, 16 (6), 3557-3562.
14. Wang, F.; Zahid, O. K.; Swain, B. E.; Parsonage, D.; Hollis, T.; Harvey, S.; Perrino, F. W.; Kohli, R. M.; Taylor, E. W.; Hall, A. R., Solid-State Nanopore Analysis of Diverse DNA Base Modifications Using a Modular Enzymatic Labeling Process. Nano Letters 2017, 17 (11), 7110-7116.
15. Yusko, E. C.; Bruhn, B. R.; Eggenberger, O. M.; Houghtaling, J.; Rollings, R. C.; Walsh, N. C.; Nandivada, S.; Pindrus, M.; Hall, A. R.; Sept, D.; Li, J.; Kalonia, D. S.; Mayer, M., Real-Time Shape Approximation and Fingerprinting of Single Proteins Using a Nanopore. Nature Nanotechnology 2017, 12 (4), 360-367.
16. Charron, M.; Briggs, K.; King, S.; Waugh, M.; Tabard-Cossa, V., Precise DNA Concentration Measurements with Nanopores by Controlled Counting. Analytical Chemistry 2019, 91 (19), 12228-12237.
17. Carter, J. M.; Hussain, S., Robust Long-Read Native DNA Sequencing Using the ONT CsgG Nanopore System. Wellcome Open Research 2017, 2, 23.
18. Noakes, M. T.; Brinkerhoff, H.; Laszlo, A. H.; Derrington, I. M.; Langford, K. W.; Mount, J. W.; Bowman, J. L.; Baker, K. S.; Doering, K. M.; Tickman, B. I.; Gundlach, J. H., Increasing the Accuracy of Nanopore DNA Sequencing Using a Time-Varying Cross Membrane Voltage. Nature Biotechnology 2019, 37 (6), 651-656.
19. Ouldali, H.; Sarthak, K.; Ensslen, T.; Piguet, F.; Manivet, P.; Pelta, J.; Behrends, J. C.; Aksimentiev, A.; Oukhaled, A., Electrical Recognition of the Twenty Proteinogenic Amino Acids Using an Aerolysin Nanopore. Nature Biotechnology 2020, 38 (2), 176-181.
20. Li, J.; Stein, D.; McMullan, C.; Branton, D.; Aziz, M. J.; Golovchenko, J. A., Ion-Beam Sculpting at Nanometre Length Scales. Nature 2001, 412 (6843), 166-169.
21. Chen, P.; Mitsui, T.; Farmer, D. B.; Golovchenko, J.; Gordon, R. G.; Branton, D., Atomic Layer Deposition to Fine-Tune the Surface Properties and Diameters of Fabricated Nanopores. Nano Letters 2004, 4 (7), 1333-1337.
22. Storm, A. J.; Chen, J. H.; Zandbergen, H. W.; Dekker, C., Translocation of Double-Strand DNA through a Silicon Oxide Nanopore. Phys Rev E Stat Nonlin Soft Matter Phys 2005, 71 (5 Pt 1), 051903.
23. Garaj, S.; Hubbard, W.; Reina, A.; Kong, J.; Branton, D.; Golovchenko, J. A., Graphene as a Subnanometre Trans-Electrode Membrane. Nature 2010, 467 (7312), 190-193.
24. Larkin, J.; Henley, R.; Bell, D. C.; Cohen-Karni, T.; Rosenstein, J. K.; Wanunu, M., Slow DNA Transport through Nanopores in Hafnium Oxide Membranes. ACS Nano 2013, 7 (11), 10121-10128.
25. Liu, S.; Lu, B.; Zhao, Q.; Li, J.; Gao, T.; Chen, Y.; Zhang, Y.; Liu, Z.; Fan, Z.; Yang, F.; You, L.; Yu, D., Boron Nitride Nanopores: Highly Sensitive DNA Single-Molecule Detectors. Advanced Materials 2013, 25 (33), 4549-4554.
26. Liu, K.; Feng, J.; Kis, A.; Radenovic, A., Atomically Thin Molybdenum Disulfide Nanopores with High Sensitivity for DNA Translocation. ACS Nano 2014, 8 (3), 2504-2511.
27. Mojtabavi, M.; VahidMohammadi, A.; Liang, W.; Beidaghi, M.; Wanunu, M., Single-Molecule Sensing Using Nanopores in Two-Dimensional Transition Metal Carbide (MXene) Membranes. ACS Nano 2019, 13 (3), 3042-3053.
28. Kasianowicz, J. J.; Brandin, E.; Branton, D.; Deamer, D. W., Characterization of Individual Polynucleotide Molecules Using a Membrane Channel. Proceedings of the National Academy of Sciences 1996, 93 (24), 13770-13773.
29. Stefureac, R.; Long, Y.-t.; Kraatz, H.-B.; Howard, P.; Lee, J. S., Transport of α-Helical Peptides through α-Hemolysin and Aerolysin Pores. Biochemistry 2006, 45 (30), 9172-9179.
30. Chen, M.; Khalid, S.; Sansom, M. S. P.; Bayley, H., Outer Membrane Protein G: Engineering a Quiet Pore for Biosensing. Proceedings of the National Academy of Sciences 2008, 105 (17), 6272.
31. Derrington, I. M.; Butler, T. Z.; Collins, M. D.; Manrao, E.; Pavlenok, M.; Niederweis, M.; Gundlach, J. H., Nanopore DNA Sequencing with MspA. Proceedings of the National Academy of Sciences 2010, 107 (37), 16060.
32. Soskine, M.; Biesemans, A.; De Maeyer, M.; Maglia, G., Tuning the Size and Properties of ClyA Nanopores Assisted by Directed Evolution. Journal of the American Chemical Society 2013, 135 (36), 13456-13463.
33. Goyal, P.; Krasteva, P. V.; Van Gerven, N.; Gubellini, F.; Van den Broeck, I.; Troupiotis-Tsailaki, A.; Jonckheere, W.; Pehau-Arnaudet, G.; Pinkner, J. S.; Chapman, M. R.; Hultgren, S. J.; Howorka, S.; Fronzes, R.; Remaut, H., Structural and Mechanistic Insights into the Bacterial Amyloid Secretion Channel CsgG. Nature 2014, 516 (7530), 250-3.
34. Huang, G.; Willems, K.; Soskine, M.; Wloka, C.; Maglia, G., Electro-Osmotic Capture and Ionic Discrimination of Peptide and Protein Biomarkers with FraC Nanopores. Nature Communications 2017, 8 (1), 935.
35. Gornall, J. L.; Mahendran, K. R.; Pambos, O. J.; Steinbock, L. J.; Otto, O.; Chimerel, C.; Winterhalter, M.; Keyser, U. F., Simple Reconstitution of Protein Pores in Nano Lipid Bilayers. Nano Letters 2011, 11 (8), 3334-3340.
36. Diederichs, T.; Pugh, G.; Dorey, A.; Xing, Y.; Burns, J. R.; Hung Nguyen, Q.; Tornow, M.; Tampé, R.; Howorka, S., Synthetic Protein-Conductive Membrane Nanopores Built with DNA. Nature Communications 2019, 10 (1), 5018.
37. Yu, L.; Kang, X.; Alibakhshi, M. A.; Pavlenok, M.; Niederweis, M.; Wanunu, M., Stable Polymer Bilayers for Protein Channel Recordings at High Guanidinium Chloride Concentrations. Biophysical Journal 2021, 120 (9), 1537-1541.
38. Hall, A. R.; Scott, A.; Rotem, D.; Mehta, K. K.; Bayley, H.; Dekker, C., Hybrid Pore Formation by Directed Insertion of α-Haemolysin into Solid-State Nanopores. Nature Nanotechnology 2010, 5 (12), 874-877.
39. Cressiot, B.; Greive, S. J.; Mojtabavi, M.; Antson, A. A.; Wanunu, M., Thermostable Virus Portal Proteins as Reprogrammable Adapters for Solid-State Nanopore Sensors. Nature Communications 2018, 9 (1), 4652.
40. Cressiot, B.; Greive, S. J.; Si, W.; Pascoa, T. C.; Mojtabavi, M.; Chechik, M.; Jenkins, H. T.; Lu, X.; Zhang, K.; Aksimentiev, A.; Antson, A. A.; Wanunu, M., Porphyrin-Assisted Docking of a Thermophage Portal Protein into Lipid Bilayers: Nanopore Engineering and Characterization. ACS Nano 2017, 11 (12), 11931-11945.
41. Zemek, J.; Vystrcil, T.; Lesiak-Orlowska, B.; Jablonski, A.; Luches, A., Auger Si Lvv, O Kll and N Kll Lineshapes at Air-Exposed Silicon Nitride Surfaces: Pattern Recognition Analysis. Surface and Interface Analysis 1994, 21 (11), 771-777.
42. Wanunu, M.; Meller, A., Chemically Modified Solid-State Nanopores. Nano Letters 2007, 7 (6), 1580-1585.
43. Anderson, B. N.; Muthukumar, M.; Meller, A., pH Tuning of DNA Translocation Time through Organically Functionalized Nanopores. ACS Nano 2013, 7 (2), 1408-1414.
44. Tan, S.; Wang, L.; Liu, H.; Wu, H.; Liu, Q., Single Nanoparticle Translocation through Chemically Modified Solid Nanopore. Nanoscale Research Letters 2016, 11 (1), 50.
45. Kim, D.; Zuidema, J. M.; Kang, J.; Pan, Y.; Wu, L.; Warther, D.; Arkles, B.; Sailor, M. J., Facile Surface Modification of Hydroxylated Silicon Nanostructures Using Heterocyclic Silanes. Journal of the American Chemical Society 2016, 138 (46), 15106-15109.
46. Pan, Y.; Maddox, A.; Min, T.; Gonzaga, F.; Goff, J.; Arkles, B., Surface-Triggered Tandem Coupling Reactions of Cyclic Azasilanes. Chem Asian J 2017, 12 (11), 1198-1203.
47. Arkles, B.; Goff, J.; Min, T.; Pan, Y.; Phillips, A.; DeMella, K.; Brick, C., Single-Molecule Orthogonal Double-Click Chemistry—Inorganic to Organic Nanostructure Transition. ACS Applied Materials & Interfaces 2020, 12 (24), 27737-27744.
48. Glionna, C.; Kumar, V.; Le Saux, G.; Pramanik, B.; Wagner, N.; Cohen-Luria, R.; Ashkenasy, G.; Ashkenasy, N., Dynamic Surface Layer Coiled Coil Proteins Processing Analog-to-Digital Information. Journal of the American Chemical Society 2021, 143 (42), 17441-17451.
49. Kowalczyk, S. W.; Kapinos, L.; Blosser, T. R.; Magalhães, T.; van Nies, P.; Lim, R. Y. H.; Dekker, C., Single-Molecule Transport across an Individual Biomimetic Nuclear Pore Complex. Nature Nanotechnology 2011, 6 (7), 433-438.
50. Wei, R.; Gatterdam, V.; Wieneke, R.; Tampé, R.; Rant, U., Stochastic Sensing of Proteins with Receptor-Modified Solid-State Nanopores. Nature Nanotechnology 2012, 7 (4), 257-263.
51. Kobashi, K., Catalytic Oxidation of Sulfhydryl Groups by O-Phenanthroline Copper Complex. Biochimica et Biophysica Acta (BBA)—General Subjects 1968, 158 (2), 239-245.
52. Bayfield, O. W.; Steven, A. C.; Antson, A. A., Cryo-Em Structure in Situ Reveals a Molecular Switch That Safeguards Virus against Genome Loss. eLife 2020, 9, e55517.

### INCORPORATION BY REFERENCE; EQUIVALENTS

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of making a sensor, the method comprising:
a) reacting a membrane, which comprises a nanopore and a silicon nitride surface, with 2,2-dimethoxyl-1-thia-2-silacyclopentane, thereby forming a thiol-silane surface on the membrane comprising the nanopore; and
b) contacting the membrane comprising the nanopore and the thiol-silane surface with a portal protein in the presence of Cu(phenanthroline)₂, thereby forming a disulfide bond between a cysteine residue of the portal protein and the thiol-silane surface of the membrane comprising the nanopore.

2. The method of claim 1, wherein the disulfide bond is between the cysteine residue of the portal protein and the thiol-silane surface of the nanopore portion of the membrane.

3. The method of claim 1, wherein reacting the membrane comprising the nanopore and the silicon nitride surface with 2,2-dimethoxyl-1-thia-2-silacyclopentane occurs in the presence of a polar aprotic solvent.

4. The method of claim 3, wherein the polar aprotic solvent is dicholoromethane.

5. The method of claim 1, wherein the portal protein is hydrophilic.

6. The method of claim 5, wherein the portal protein comprises a G20c mutant comprising at least one amino acid substitution of an externally facing leucine to a cysteine.

7. The method of claim 6, wherein the portal protein comprises a G20c mutant comprising at least one amino acid substitution from a negatively charged amino acid to a polar amino acid.

8. The method of claim 7, wherein the amino acid substitution is from aspartic acid to asparagine.

9. The method of claim 5, wherein the portal protein is a G20c mutant in which four aspartic acid residues are substituted with asparagine.

10. The method of claim 9, wherein the portal protein comprises SEQ ID NO: 3.

11. The method of claim 10, further comprising rinsing the membrane with solvent prior to step b).

12. The method of claim 1, wherein the silicon nitride surface of the membrane is on silicon dioxide.

13. The method of claim 12, further comprising making a nanopore in the membrane by transmission electron microscopy.

14. The method of claim 13, wherein the nanopore diameter is from 7 nm to 8 nm.

15. The method of claim 13, further comprising immersing the membrane in a mixture of sulfuric acid and hydrogen peroxide after making the nanopore in the membrane.

16. (canceled)

17. The method of claim 15, further comprising washing the membrane with hot water after immersion in the mixture of sulfuric acid and hydrogen peroxide.

18. (canceled)

19. The method of claim 14, further comprising drying the membrane.

20. (canceled)

21. The method of claim 19, wherein drying further comprises baking the membrane at a temperature from 130° C. to 170° C. for duration from 3 minutes to 5 minutes.

22. A sensor comprising:
a membrane comprising a nanopore with a portal protein through the nanopore, wherein the membrane is covalently bonded to the portal protein by a disulfide bond between a cysteine residue of the portal protein and a thiol-silane surface of the membrane.

23-34. (canceled)

35. A method for sensing an analyte comprising:
a) providing a sensor comprising a membrane, which comprises a thiol-silane surface and a nanopore, with a portal protein through the nanopore, wherein the membrane is covalently bonded to the portal protein by a disulfide bond between a cysteine residue of the portal protein and the thiol-silane surface of the membrane;
b) providing a first conductive liquid and a second conductive liquid, wherein the portal protein comprises a tunnel that provides liquid communication between the first conductive liquid and second conductive liquid;
c) applying an electrical potential between the first conductive liquid medium and the second conductive liquid medium to establish flow of electrical current through the tunnel of the portal protein;
d) contacting the sensor with an analyte; and
e) measuring a change in flow of electrical current through the tunnel of the portal protein indicative of translocation of the analyte, or a portion of the analyte, through the tunnel of the portal protein.

36-42. (canceled)


SEQUENCE LISTING (4 sequences)

SEQ ID NO: 1 - protein, 448 aa, Thermus thermophilus
MAKRGRKPKELVPGPGSIDPSDVPKLEGASVPVMSTSYDVVVDREFDELLQGKDGLLVYHKMLSDGTVKNALNYIFGRIRSAKWYVEPASTDPEDIAIAAFIHAQLGIDDASVGKYPFGRLFAIYENAYIYGMAAGEIVLTLGADGKLILDKIVPIHPFNIDEVLYDEEGGPKALKLSGEVKGGSQFVSGLEIPIWKTVVFLHNDDGSFTGQSALRAAVPHWLAKRALILLINHGLERFMIGVPTLTIPKSVRQGTKQWEAAKEIVKNFVQKPRHGIILPDDWKFDTVDLKSAMPDAIPYLTYHDAGIARALGIDFNTVQLNMGVQAINIGEFVSLTQQTIISLQREFASAVNLYLIPKLVLPNWPSATRFPRLTFEMEERNDFSAAANLMGMLINAVKDSEDIPTELKALIDALPSKMRRALGVVDEVREAVRQPADSRYLYTRRRR

SEQ ID NO: 2 - DNA, 1347 bp, Thermus thermophilus
  source 1..1347: mol_type = genomic DNA
atggctaagcgaggacgtaaacccaaagagctggtccccggacctggctccattgacccatctgacgttcccaagctcgagggcgcctccgtgccggtgatgtccaccagttacgacgtggtggtggaccgggagtttgacgagctactgcagggcaaggacggcttgctcgtctaccacaagatgctctcggacggcacggttaagaacgccctcaactacatcttcggacgcatccgctcggcgaagtggtacgtagagcccgcctctaccgacccggaagacatcgccatcgccgccttcatccacgcccagttaggcatagacgacgcttcggtgggcaagtatccctttggccgccttttcgccatctacgaaaacgcctacatatacggcatggccgccggggaaatcgtactaacccttggcgcggacggcaagctcatccttgacaaaatcgtccctatccaccctttcaacattgacgaggtgctttacgacgaggaaggcggtccaaaggcgctaaagctaagcggagaggtgaagggcggaagccagtttgtgagcgggttggagattcctatatggaagaccgtggtcttcctgcacaacgacgacggctccttcaccggacagagcgccctcagagccgccgtgccgcattggctagccaaacgagccctcattctcctcatcaaccacgggttggagcgcttcatgattggcgtgcccaccctcaccatccccaagagcgtgcgtcagggaaccaagcaatgggaggccgccaaggaaatcgtcaagaactttgttcaaaaaccacggcatggtataatactgcctgacgactggaagtttgacacggtagacctgaagtcggccatgcccgacgccattccctacctgacctaccacgacgcgggcatcgctagggcgcttggcatagacttcaacaccgttcaactaaacatgggggtacaggcgataaacatcggcgagttcgtaagcctgacccagcagaccatcatttcgctccagcgggagttcgctagcgcggtcaacctctacctcatccccaagctagtgcttcccaactggccgagcgctactcgctttcctaggctcacctttgagatggaggagcgcaacgacttctccgccgcggccaaccttatgggcatgctcatcaacgcggttaaggactccgaagacattcccaccgagctcaaggcgctaatagacgctctgcctagcaagatgcgccgggcgcttggcgtggtggacgaggtgagggaagcggtacgccaacccgccgattcccgctacctgtacacgcgaaggaggaggtag

SEQ ID NO: 3 - protein, 436 aa, synthetic construct
  REGION 1..417: note = CD/N G20c mutant
MGSSHHHHHHSSGLEVLFQGPAKLEGASVPVMSTSYDVVVDREFDECLQGKDGLLVYHKMLSDGTVKNALNYIFGRIRSAKWYVEPASTDPEDIAIAAFIHAQLGIDDASVGKYPFGRLFAIYENAYIYGMAAGEIVLTLGADGKLILDKIVPIHPFNIDEVLYDEEGGPKALKLSGEVKGGSQFVSGLEIPIWKTVVFLHNDDGSFTGQSALRAAVPHWLAKRALILLINHGLERFMIGVPTLTIPKSVRQGTKQWEAAKEIVKNFVQKPRHGIILPNDWKFNTVNLKSAMPNAIPYLTYHDAGIARALGIDFNTVQLNMGVQAINIGEFVSLTQQTIISLQREFASAANLYLIPKLVLPNWPSATRFPRLTFEMEERNDFSAAANLMGMLINAVKDSEDIPTELKALIDALPSKMRRALGVVDEVREAVRQPAD

SEQ ID NO: 4 - DNA, 1311 bp, synthetic construct
  misc_feature 1..1311: note = CD/N G20c mutant
  source 1..1311: mol_type = other DNA
atgggcagcagccatcatcatcatcatcacagcagcggcctggaagttctgttccagggaccagcaaagctcgagggcgcctccgtgccggtgatgtccaccagttacgacgtggtggtggaccgggagtttgacgagtgtctgcagggcaaggacggcttgctcgtctaccacaagatgctctcggacggcacggttaagaacgccctcaactacatcttcggacgcatccgctcggcgaagtggtacgtagagcccgcctctaccgacccggaagacatcgccatcgccgccttcatccacgcccagttaggcatagacgacgcttcggtgggcaagtatccctttggccgccttttcgccatctacgaaaacgcctacatatacggcatggccgccggggaaatcgtactaacccttggcgcggacggcaagctcatccttgacaaaatcgtccctatccaccctttcaacattgacgaggtgctttacgacgaggaaggcggtccaaaggcgctaaagctaagcggagaggtgaagggcggaagccagtttgtgagcgggttggagattcctatatggaagaccgtggtcttcctgcacaacgacgacggctccttcaccggacagagcgccctcagagccgccgtgccgcattggctagccaaacgagccctcattctcctcatcaaccacgggttggagcgcttcatgattggcgtgcccaccctcaccatccccaagagcgtgcgtcagggaaccaagcaatgggaggccgccaaggaaatcgtcaagaactttgttcaaaaaccacggcatggtataatactgcctaacgactggaagtttaacacggtaaacctgaagtcggccatgcccaacgccattccctacctgacctaccacgacgcgggcatcgctagggcgcttggcatagacttcaacaccgttcaactaaacatgggggtacaggcgataaacatcggcgagttcgtaagcctgacccagcagaccatcatttcgctccagcgggagttcgctagcgcggccaacctctacctcatccccaagctagtgcttcccaactggccgagcgctactcgctttcctaggctcacctttgagatggaggagcgcaacgacttctccgccgcggccaaccttatgggcatgctcatcaacgcggttaaggactccgaagacattcccaccgagctcaaggcgctaatagacgctctgcctagcaagatgcgccgggcgcttggcgtggtggacgaggtgagggaagcggtacgccaacccgccgattaa